(19) United States
(12) Patent Application Publication
(10) Pub. No.: US 2013/0184678 A1
(43) Pub. Date: Jul. 18, 2013

(54) DUAL ACTION SYRINGE

(71) Applicants: Mohiddin Rida Dimashkieh, Riyadh (SA); Mohammad Ibrahim Al Obaida, Riyadh (SA)

(72) Inventors: Mohiddin Rida Dimashkieh, Riyadh (SA); Mohammad Ibrahim Al Obaida, Riyadh (SA)

(73) Assignee: KING SAUD UNIVERSITY, RIYADH (SA)

(21) Appl. No.: 13/822,627

(22) PCT Filed: Oct. 11, 2010
(86) PCT No.: PCT/US2010/052195

§ 371 Date: Mar. 12, 2013

(51) Int. Cl. A61M 5/42

(52) U.S. Cl. CPC A61M 5/422
USPC 604/506; 604/112; 29/428

(57) ABSTRACT

In one aspect, a dual action syringe includes an external body having a distal end defining an anesthetic gel receptive concavity surrounding a needle passage through the distal end. The external body also includes a proximal end defining a plunger receptive passage. An internal body is slidably disposed in the external body. The internal body receives a needle in a distal end of the internal body and an anesthetic carpule to provide fluid flow communication between the carpule and the needle in response to pressure on the plunger. A biasing member is disposed in the external body, between the external body and the internal body, biasing a distal end of the needle away from the distal end of the external body into the external body; whereby, pressure on the plunger first moves the needle through the needle passage and subsequently provides fluid flow communication between the carpule and the needle.

### BACKGROUND

In dentistry and medicine, the use of topical anesthetic gel prior to needle insertion into mucous membrane is common. When a topical anesthetic gel is applied properly, the administration of local anesthesia using a syringe needle can be made with minimum pain and discomfort. To insure proper use and effective application topical anesthetic gel is typically applied by carrying a small amount of the anesthetic gel on a cotton applicator to the site to be injected and applying the gel on the injection area for at least one minute, prior to injection of the anesthetic.

Typical sleeve protected needles, such as may be used for application of local anesthesia, are manually retracted in order to protect the doctor, the patient and the medical staff from needle stick injuries during deployment or withdrawal of the needle, and manually extended to inject the anesthesia. These existing sleeve protected needles are bulky.

### SUMMARY

The described systems and methods relate to a dual action syringe that includes an external body having a distal end defining an anesthetic gel receptive concavity surrounding a needle passage through the distal end. The external body also includes a proximal end defining a plunger receptive passage. An internal body is slidably disposed in the external body. The internal body receives a needle in a distal end of the internal body and an anesthetic carpule to provide fluid flow communication between the carpule and the needle in response to pressure on the plunger. A biasing member is disposed in the external body, between the external body and the internal body, biasing a distal end of the needle away from the distal end of the external body, into the external body. Whereby, pressure on the plunger first moves the needle through the needle passage and subsequently provides fluid flow communication between the carpule and the needle.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

In the Figures, the left-most digit of a component reference number identifies the particular Figure in which the component first appears.

FIG. 1 is a side view of an example of the present dual action syringe, with its needle retracted, according to one embodiment.

FIG. 2 is a distal end view of the example of the dual action syringe of FIG. 1, according to one embodiment.

FIG. 3 is a side view of the example of the dual action syringe of FIGS. 1 and 2, with the needle deployed, according to one embodiment.

FIG. 4 shows a fragmented, generally cross-sectional view of the example of the example dual action syringe of FIGS. 1 through 3, with its needle retracted, according to one embodiment.

FIG. 5 shows a fragmented, generally cross-sectional view of the example dual action syringe of FIGS. 1 through 4, with the needle deployed, according to one embodiment.

FIG. 6 shows a fragmented, generally cross-sectional exploded view of the example dual action syringe of FIGS. 1 through 5, according to one embodiment.

FIG. 7 shows an example procedure for assembly of a dual action syringe, according to one embodiment.

FIG. 8 shows an example procedure for use of a dual action syringe, according to one embodiment.

### DETAILED DESCRIPTION

### Overview

The systems and methods described herein relate to a dual action syringe used to apply topical anesthesia on soft tissues prior to needle insertion, which significantly reduces pain associated with needle insertion. The present dual action syringe also secures the needle to aid in preventing stick injuries to the doctor, the patient and the medical staff during deployment or withdrawal of the needle. In accordance with various implementations, the needle is not visible to the patient during deployment, and thus may help reduce the patient's fear and anxiety during the visit, particularly child patients.

The present dual action syringe allows a smooth automatic retraction of the needle into the body of the syringe, without the need to pull the syringe plunger. This overcomes shortcomings of prior sleeve protected needles. Although such prior needles can be retracted, these devices do not provide automatic instantaneous retraction of the needle. In the present dual action syringe, needle retraction is automatic and is carried out by a built-in spring.

In certain implementations, the dual action syringe has an external body, which is generally cylindrical and may be threaded on each end. On the proximal end (relative to the user) of the external body, an end carries the plunger of the syringe and the distal end is pierced by a bore/channel, through which the needle slides. The distal end also defines a depression, referred to herein as a “concavity,” which may carry the anesthetic gel to the injection site.

In such implementations, an internal body of the present dual action syringe is a smaller generally cylindrical body or frame-like structure that is threaded at the distal end to accept the needle. This internal body can slide freely inside the external body. The internal body also operatively receives and secures a typical 1.8 ml anesthetic carpule or the like for expression of the anesthetic through the needle. A carpule is a type of ampule or cartridge containing liquid medication to be inserted using a syringe. It is often a tube with a puncturable cap on a distal end and a sliding plug on the other end.

A spring is deployed in the external body of the dual action syringe. This spring pushes the internal part of the syringe toward the proximal end of the dual action syringe, automatically, when finger pressure on the plunger is released, thus securing the needle inside the body of the syringe.

The present dual action syringe can apply topical anesthetic gel before introducing a needle into tissue. Due to concentric arrangement of the needle and the concavity holding the gel, the topical anesthetic gel is accurately applied to the area to be injected. The present dual action syringe helps in overcoming patient fear and anxiety associated with seeing the needle before and after injection. The present dual action syringe also aids in time and procedure management by streamlining steps associated with persuading a patient to accept an injection and applying topical anesthetic gel. The present dual action syringe helps prevent accidental patient injury, such as might occur to the lips tongue or other soft tissues in the mouth when carrying syringe into patients' mouth in dental procedures, as well lessening the possibility of accidental needle prick injuries to medical and personnel, thus preventing the incidence of transmission of dangerous communicable diseases such as AIDS and Hepatitis. The present dual action syringe is extremely safe to use, even in hands of inexperienced students, interns, and/or the like. Further, the present dual action syringe helps to reduce pain during injection as it allows local anesthesia drops to seep into soft tissue during needle insertion. The present dual action syringe does not require special needles or anesthetic carpules. Generally, available needles and local anesthetic carpules can be used with the present dual action syringe.

Particular examples of the present dual action syringe are generally discussed herein with respect to dental procedures. However, the present dual action syringe can be utilized in medical, veterinary, and similar procedures, as well.

### An Exemplary Dual Action Syringe

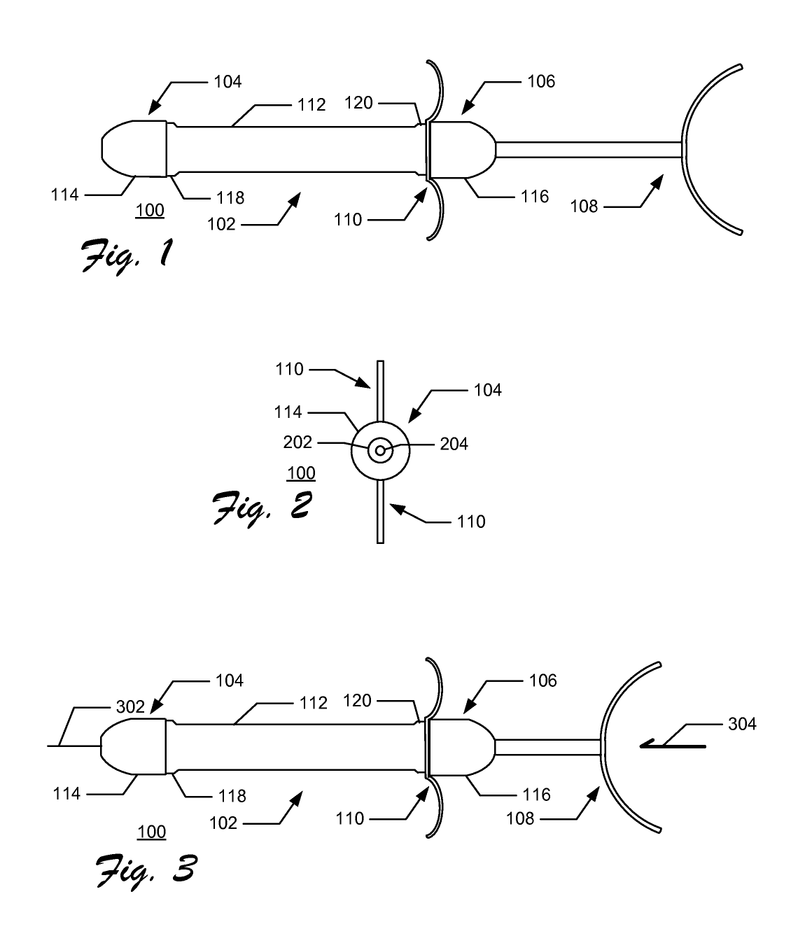

FIG. 1 is a side view of example dual action syringe 100, with its needle retracted, according to one embodiment. Dual action syringe 100 has external body 102 that includes distal end 104 defining an anesthetic receptive concavity surrounding a needle passage through the distal end. External body 102 also includes proximal end 106 defining a passage receiving plunger 108. The external body also mounts flange 110. As illustrated, external body 102 might be made up of a central and generally cylindrical tube 112 that is threaded on each end to receive a distal end cap 114 to define external body distal end 104. Central tube 112 may also threadably receive proximal end cap 116 to define external body proximal end 106. Circumferential stops 118 and 120 may be defined on central tube 112, adjacent to respective cap threads to arrest threading of caps 114 and/or 116, and/or to provide a bearing surface to secure flange 110, between proximal end cap 116 and stop 120.

FIG. 2 is a distal end view of example dual action syringe 100 of FIG. 1, according to one embodiment. Anesthetic gel receptive concavity 202 is shown surrounding needle passage 204 through distal end 104 of external body 102. As illustrated in FIG. 2, concavity 202 may be generally concentric with needle passage 204. This aids in delivery of the anesthetic gel to the intended injection site.

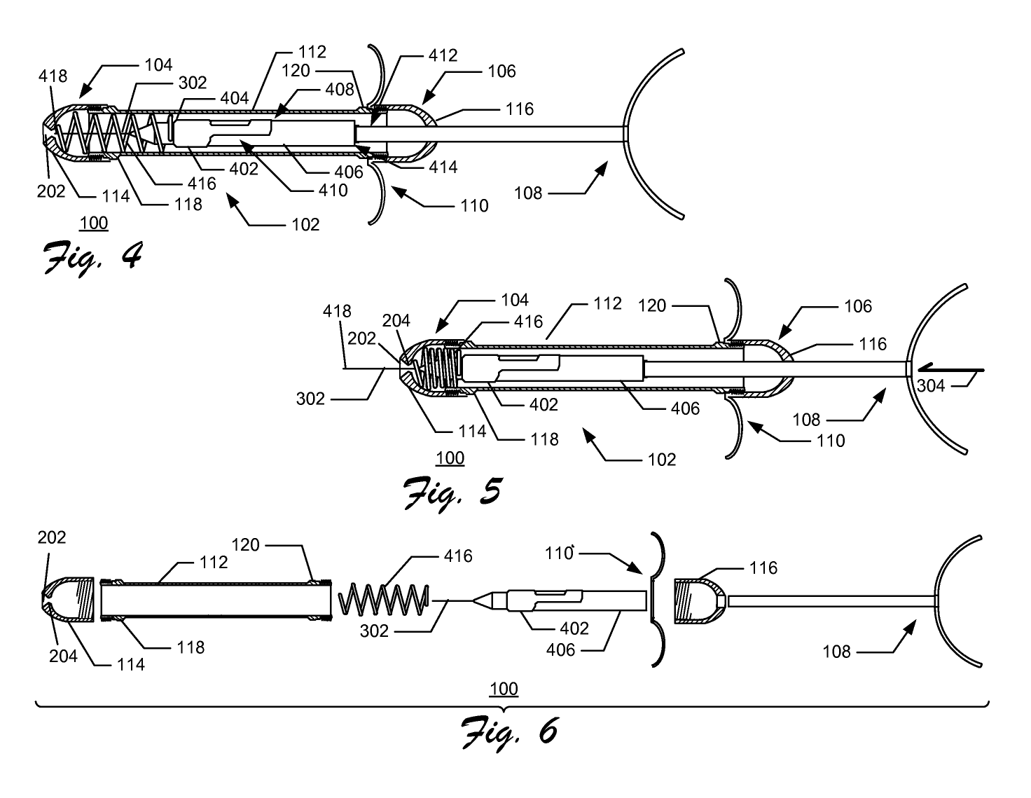

FIG. 3 is a side view of example dual action syringe 100 of FIGS. 1 and 2, with needle 302 deployed, according to one embodiment. In FIG. 3, plunger 108 is depressed by pressure generally indicated by arrow 304 to deploy needle 302. When pressure 304 on plunger 108 is released needle 302 will retract into external body 102 as shown in FIG. 1.

FIG. 4 shows a fragmented, generally cross-sectional view of example dual action syringe 100 of FIGS. 1 through 3, with needle 302 retracted, according to one embodiment. In FIG. 4, internal body 402 is shown slidably disposed in external body 102. Internal body 402 may be generally tubular and/or may have a generally cylindrical framework-like structure, as illustrated in FIG. 4. Internal body 402 receives needle 302 in distal end 404. For example, needle 302 may be screwed into distal end 404 of internal body 402. Internal body 402 also receives anesthetic carpule 406, such as through illustrated open proximal end 408 and/or open side 410. Distal end 412 of plunger 108 is received in proximal end 414 of carpule 406. Thus deployed, fluid flow communication between carpule 406 and needle 302 occurs in response to pressure 304 on plunger 108. A biasing member, such as illustrated coil spring 416 is disposed in external body 102, such as between external body 102 and internal body 402, biasing distal end of needle 302 away from distal end 104 of external body 102, into external body 102. As shown in FIG. 4, coil spring 416 may be disposed between an inner surface of external body 102, such as an inside surface of distal end cap 114, to bias distal end 418 of needle 302 away from distal end 104 of external body 102, into external body 102.

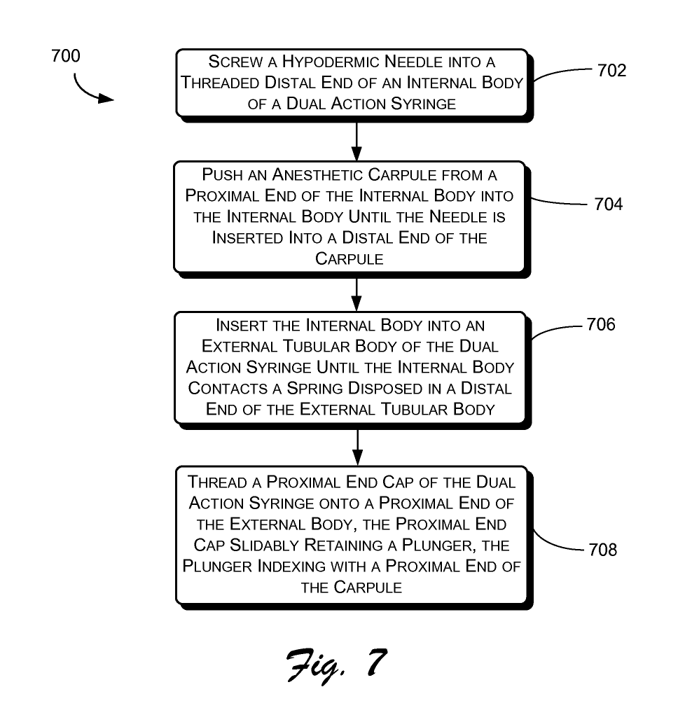

FIG. 5 shows a fragmented, generally cross-sectional view of example dual action syringe 100 of FIGS. 1 through 4, with the needle deployed, according to one embodiment. Therein, pressure 304 on plunger 108 first moves distal end 418 of needle 302 into, and out of, needle passage 204, compressing spring 416. Further pressure 304 subsequently provides fluid flow communication of anesthetic in carpule 406, between carpule 406 and needle 302.

FIG. 6 shows a fragmented, generally-cross sectional exploded view of example dual action syringe 100 of FIGS. 1 through 5, according to one embodiment.

### An Exemplary Procedure for Assembling a Dual Action Syringe

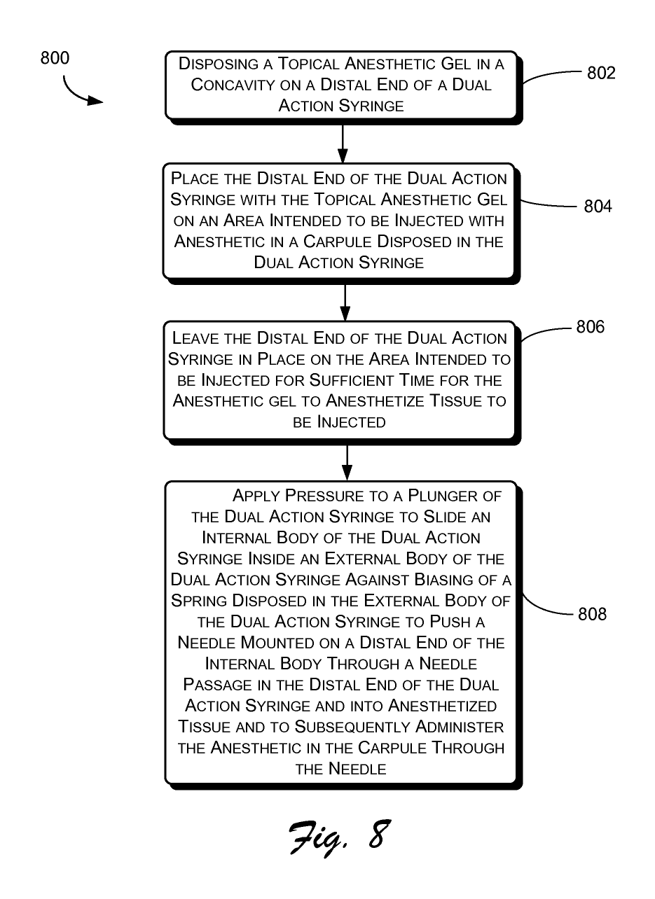

FIG. 7 shows example procedure 700 for assembly of a dual action syringe, according to one embodiment. To assemble various embodiments of the present dual action syringe, a syringe needle first is screwed into a threaded distal end of an internal body of the dual action syringe, per step 702. At 704, an anesthetic carpule is pushed from the proximal end of the internal body into the internal body, until the needle is inserted freely into the carpule's puncturable cap and secured. The assembled internal body then is inserted into the proximal end of the external body of the dual action syringe at 706. A distal end cap defining a central needle passage and a concavity, concentric with the needle passage, may have already been threaded onto the external body. Regardless, at 706, the internal body is inserted into the external body until the internal body contacts a spring disposed in a distal end of the external body. Then at 708, a proximal end cap, which carries a plunger, is inserted on the proximal end of the external body and screwed on. The internal body now is operatively deployed inside the external body of the syringe between the plunger and the spring. Thereby, the syringe is ready for use.

### An Exemplary Procedure for Using a Dual Action Syringe

FIG. 8 shows example procedure 800 for use of a dual action syringe, according to one embodiment. After syringe assembly is complete, such as described above with respect to FIG. 7, an appropriate amount of topical anesthetic gel is placed in the needle passage concentric concavity on the anterior part of the syringe, at 802. Then at 804, the anterior distal tip of the syringe, with the topical anesthetic gel, is placed on the area intended to be injected. The anterior distal end of the syringe is left in place at 806, allowing sufficient contact time (at least one minute) for the gel with soft tissues at the injection site to anesthetize the tissue to be injected. During step 806, the distal end of the syringe may be pressed against the area to be injected to aid efficiency of the topical anesthesia gel. Then at 808, light thumb pressure is applied on the syringe plunger, which slides the internal part of the syringe inside the external body, pressing on the spring disposed therebetween, which in turn pushes the needle through the needle passage in the distal end of the syringe into the soft tissue into which the local anesthetic solution is administered. When finished, the thumb pressure may be released from the plunger, allowing automatic retraction of the needle, back into the external body of the syringe, which secures and hides the needle as it is taken away from the injection site.

### Conclusion

Although a dual action syringe has been described in language specific to structural features and/or methodological operations or actions, it is understood that the implementations defined in the appended claims are not necessarily limited to the specific features or actions described. Rather, the specific features and operations of a dual action syringe are disclosed as exemplary forms of implementing the claimed subject matter.

1. A dual action syringe comprising:
an external body having a distal end defining an anesthetic gel receptive concavity surrounding a needle passage through the distal end, the external body further comprising a proximal end defining a plunger receptive passage;
an internal body slidably disposed within the external body, the internal body receiving a needle in a distal end of the internal body and receiving an anesthetic carpule to provide fluid flow communication between the carpule and the needle in response to pressure on the plunger; and
a biasing member disposed within the external body, between the external body and the internal body, biasing a distal end of the needle away from the distal end of the external body, into the external body, whereby pressure on the plunger first moves the needle into the needle passage and subsequently provides the fluid flow communication between the carpule and the needle.

2. The dual action syringe of claim 1 wherein the concavity is generally concentric with the needle passage.

3. The dual action syringe of claim 1 wherein the external body comprises a central tube threadably receiving a distal end cap defining the external body distal end.

4. The dual action syringe of claim 1 wherein the external body comprises a central tube threadably receiving a proximal end cap defining the external body proximal end.

5. The dual action syringe of claim 1 wherein the plunger is received in a proximal end of the carpule.

6. The dual action syringe of claim 1 wherein the internal body is generally tubular.

7. The dual action syringe of claim 1 wherein the internal body comprises a generally cylindrical framework.

8. The dual action syringe of claim 1 wherein the biasing member comprises a coil spring disposed between an inner surface of the external body, the distal end of the internal body to bias the distal end of the needle away from the distal end of the external body, into the external body.

9. The dual action syringe of claim 1 wherein the biasing member comprises a coil spring disposed between an inner surface of the distal end of the external body, the distal end of the internal body to bias the distal end of the needle away from the distal end of the external body, into the external body.

10. A method comprising:
screwing a syringe needle into a threaded distal end of an internal body of a dual action syringe;
pushing an anesthetic carpule from a proximal end of the internal body into the internal body, until the needle is inserted into a distal end of the carpule;
inserting the internal body into an external tubular body of the dual action syringe until the internal body contacts a spring disposed in a distal end of the external tubular body; and
threading a proximal end cap of the dual action syringe onto a proximal end of the external body, the proximal end cap slidably retaining a plunger, the plunger indexing with a proximal end of the carpule.

11. The method of claim 10, further comprising threading a distal end cap comprising a needle passage onto a central tube comprising the external body.

12. The method of claim 11 wherein the distal end cap further comprises a concavity, concentric with the needle passage.

13. A method comprising:
disposing a topical anesthetic gel in a concavity on a distal end of a dual action syringe;
placing the distal end of the dual action syringe with the topical anesthetic gel on an area intended to be injected with anesthetic in a carpule disposed in the dual action syringe;
leaving the distal end of the dual action syringe in place on the area intended to be injected for sufficient time for the anesthetic gel to anesthetize tissue to be injected; and
applying pressure to a plunger of the dual action syringe to slide an internal body of the dual action syringe inside an external body of the dual action syringe, against biasing of a spring disposed in the external body of the dual action syringe to push a needle mounted on a distal end of the internal body through a needle passage in the distal end of the dual action syringe and into anesthetized tissue and to subsequently administer the anesthetic in the carpule through the needle.

14. The method of claim 13 wherein the concavity is concentric with the needle passage.

15. The method of claim 13 wherein the leaving further comprises pressing the distal end of the syringe against the area to be injected.

16. The method of claim 13, further comprising:
releasing the pressure to the plunger, allowing retraction of the needle under the biasing of the spring into the external body of the dual action syringe.

17. The method of claim 13 wherein the sufficient time for the anesthetic gel to anesthetize the tissue to be injected is about one minute.
